(19) United States
(12) Patent Application Publication
(10) Pub. No.: US 2021/0310070 A1
(43) Pub. Date: Oct. 7, 2021

(54) ASSESSING INFECTION RISK TO HUMANS

(71) Applicant: Phylagen, Inc., San Francisco, CA (US)

(72) Inventors: Harrison F. Dillon, San Francisco, CA (US); Jessica L. Green, Berkeley, CA (US); Brad Taft, San Francisco, CA (US); Harith Humadi, San Francisco, CA (US); Eric Berlow, San Francisco, CA (US); Lara Reichmann, San Francisco, CA (US)

(21) Appl. No.: 17/221,672

(22) Filed: Apr. 2, 2021

Related U.S. Application Data
(60) Provisional application No. 63/005,298, filed on Apr. 4, 2020.

(51) Int. Cl. C12Q 1/6883; G16B 20/20; G16B 30/10; G16B 40/20; G16B 50/30; G16H 10/40; G16H 50/20

(52) U.S. Cl. CPC C12Q 1/6883; G16B 20/20; G16B 30/10; G16H 50/20; G16B 50/30; G16H 10/40; G16B 40/20

(57) ABSTRACT

The present disclosure provides methods for assessing the risk of infection to humans posed by a surface or air in a built environment or on an object. In addition to testing for the presence of infectious agents, the methods include the generation and analyses of microbiomes, and the use of both in risk assessment.

### CROSS-REFERENCE TO RELATED APPLICATIONS

This application claims the benefit of U.S. provisional application No. 63/005,298, filed Apr. 4, 2020, which is hereby incorporated by reference in its entirety.

### INCORPORATION BY REFERENCE OF THE SEQUENCE LISTING

This application includes a sequence listing which has been submitted electronically in ASCII format and is hereby incorporated by reference in its entirety. This ASCII copy, created on Jun. 16, 2021, is named 2021-06-16_PHYLP008US_SeqList_ST25.txt and is 3,310 bytes in size.

### FIELD

The present disclosure relates to generally to the area of assessing the risk of infection to humans in a built environment and/or with respect to an object contacted by humans.

### BACKGROUND

The rapid spread of viruses such as SARS-CoV-2, which causes the disease COVID-19, has highlighted the issue of infection risk to humans from contact with elements in built environments, such as residential (e.g., multiunit, single-family home), institutional (e.g., dormitory, prison, elder care), commercial (e.g., office building, warehouse, distribution center, factory), health care (e.g., hospital), or transportation (e.g., aircraft, cruise ship, bus) environments. Surfaces of objects that humans come into contact with frequently and/or that are routinely contacted by multiple people also pose a risk. Methods for testing for the presence of pathogens in such contexts have typically focused on the identification of a single specific pathogen.

### SUMMARY

The methods described herein incorporate the finding that humans shed (e.g., via skin, hair, exhaling, sneezing, coughing) characteristic human-associated microbes. These characteristic microbes need not be pathogenic to provide information useful in assessing the risk of human infection. This is because the amount of human-associated microbes in one or more physical samples taken from air or a surface is proportional to the degree to which humans come into contact with the surface or are present in the built environment in the case of air samples.

Various embodiments contemplated herein may include, but need not be limited to, one or more of the following:

Embodiment 1: A method of assessing the risk of infection to humans in a built environment, the method comprising: (a) obtaining one or more physical samples from a surface and/or air in the built environment; (b) determining from the one or more physical samples if one or more infectious agents are present in the built environment; (c) generating a microbiome signature from the one or more physical samples and determining from the signature the amount of human-associated microbes; and (d) based on the results of steps (b) and (c), assigning a level of risk of infection to one or more humans for occupying or transiting through said built environment or any sub-area of said built environment.

Embodiment 2: The method of embodiment 1, wherein the one or more physical samples are air samples.

Embodiment 3: The method of embodiment 1, wherein the infection is selected from the group consisting of bacterial, viral, fungal, protozoal, and parasitic infections.

Embodiment 4: The method of embodiment 3, wherein the infection comprises an infection caused by a viral type selected from the group consisting of hemorrhagic viruses, respiratory viruses, gastrointestinal viruses, exanthematous viruses, hepatic viruses, cutaneous viruses, and viruses that cause neurologic disease.

Embodiment 5: The method of embodiment 4, wherein step (b) comprises performing an assay to detect the presence of at least one virus selected from the group consisting of ebola virus; dengue virus; novavirus; viruses that cause Lassa fever, yellow fever, Marburg hemorrhagic fever, and Crimean-Congo hemorrhagic fever; rhinovirus; coronavirus; adenovirus; influenza virus; parainfluenza virus; respiratory syncytial virus; enterovirus; norovirus; rotavirus; astrovirus, viruses that cause measles, rubella, chickenpox/shingles, roseola, smallpox, and fifth disease; chikungunya virus; hepatitis virus; herpesvirus, papilloma virus; molluscum contagionsum; polio virus; rabies virus; and viruses that cause viral meningitis and encephalitis.

Embodiment 6: The method of embodiment 5, wherein the virus is selected from the group consisting of: H1N1, H1N2, H2N2, H2N3, H3N1, H3N2, H3N8, H5N1, H5N2, H5N3, H5N6, H5N8, H5N9, H6N1, H6N2, H7N1, H7N2, H7N3, H7N4, H7N7, H7N9, H9N2, H10N7, H10N8, H11N2, H11N9, H17N10, H18N11, HPIV-1, HPIV-2, HPIV-3, HPIV-4, HAdV-B, HAdV-C, 229E, OC43, NL63, HUK1, SARS-CoV-2, MERS-CoV, SARS-CoV, Sin Nombre orthohantavirus, Black Creek Canal orthohantavirus, Puumala virus, Thaland virus; HRV-A1, HRV-A2, HRV-A7-13, HRV-A15, HRV-A16, HRV-A18-25, HRV-A28-34, HRV-A36, HRV-A38-41, HRV-A43-47, HRV-A49-51, HRV-A53-68, HRV-A71, HRV-A73-78, HRV-A80-82, HRV-A85, HRV-A88-90, HRV-A94-96, HRV-A98, HRV-A100-103, HRV-B3-6, HRV-B14, HRV-B17, HRV-B26, HRV-B27, HRV-B35, HRV-B37, HRV-B42, HRV-B48, HRV-B52, HRV-B69, HRV-B70, HRV-B72, HRV-B79, HRV-B83, HRV-B84, HRV-B86, HRV-B91-93, HRV-B97, HRV-B99, and HRV-C1-51.

Embodiment 7: The method of embodiment 1, wherein step (c) comprises determining the presence and relative abundance of at least one human-associated microbe selected from the group consisting of: Actinomyces, Aerococcus, Akkermansia, Alistipes, Alloiococcus, Anaerococcus, Anaerotruncus, Atopobium, Bacteroides, Barnesiella, Bifidobacterium, Blautia, Butyrivibrio, Chlamydia, Clostridium, Corynebacterium, crAssphage, Cutibacterium (formerly Propionibacterium), Dialister, Dysgonomonas, Enterobacter, Enterococcus, Escherichia, Faecalibacterium, Fusobacterium, Gardnerella, Gemella, Haemophilus, Klebsiella, Kocuria, Lactobacillus, Lactococcus, Megasphera, Methanobrevibacter, Micrococcus, Mobiluncus, Moraxella, Mycobacterium, Mycoplasma, Neisseria, Oxalobacter, Papillibacter, Parabacteriodes, Parvimonas, Peptoniphilus, Peptostreptococcus, Porphyromonas, Prevotella, Pseudomonas, Roseburia, Ruminococcus, Sneathia, Spirochaeta, Staphylococcus, Streptococcus, Villonella, Alternaria, Aspergillus, Candida, Cladosporium, Curvularia, Embellisia, Fusarium, Penicillium, Saccharomyces, Stachybotrys, Thermomyces, Trichophyton, Malassezia, and Rhodotorula.

Embodiment 8: The method of embodiment 2, wherein step (c) comprises performing qPCR to determine the presence of one or more human respiratory tract microbes selected from the group consisting of Firmicutes, Actinobacteria, Proteobacteria, Staphylococcus epidermidis, viridans group streptococci (VGS), Corynebacterium spp. (diphtheroids), Propionibacterium spp., Haemophilus spp. Prevotella, Fusobacterium, Moraxella, Candida, Pseudomonas, Streptococcus, Prevotella, Fusobacterium, and Veillonella.

Embodiment 9: The method of embodiment 1, wherein the level of risk assigned is high if any infectious agent of step (b) is identified, and one or more interventions are selected from the group consisting of introducing unfiltered outdoor air into the built environment, venting air to outdoors instead of recycling, increasing the ratio of indoor:outdoor air, increasing the air flow, and reducing occupant density.

Embodiment 10: The method of embodiment 9, wherein the air flow is increased to at least 2, 3, 4, 5, 6, 7, 8, 9 or 10 air changes per hour.

Embodiment 11: The method of any one of the preceding embodiments, wherein the level of risk assigned is intermediate if step (b) indicates no infectious agent and step (c) indicates a level of human-associated microbes that meets or exceeds a predetermined threshold.

Embodiment 12: The method of any one of the preceding embodiments, wherein the level of risk assigned is low if step (b) indicates no infectious agent and step (c) indicates a level of human-associated microbes below a predetermined threshold.

Embodiment 13: The method of embodiment 1, wherein the level of risk of infection determines which if any interventions are performed to reduce the level of risk.

Embodiment 14: A method of assessing the risk of infection to humans in a built environment, the method comprising: (a) obtaining one or more physical samples from at least one surface in the built environment; (b) determining from the one or more physical samples if one or more infectious agents are present on the at least one surface; (c) generating a microbiome signature for the at least one surface from the one or more physical samples and determining from the signature the amount of human-associated microbes present on said surface; and (d) based on the results of steps (b) and (c), assigning a level of risk of infection to one or more humans for occupying or transiting through said built environment or any sub-area of said built environment.

Embodiment 15: The method of embodiment 14, wherein the built environment is selected from the group consisting of residential, institutional, commercial, health care, and transportation environments.

Embodiment 16: The method of embodiment 14 or embodiment 15, wherein the infection is selected from the group consisting of bacterial, viral, fungal, protozoal, and parasitic infections.

Embodiment 17: The method of embodiment 16, wherein the infection comprises an infection caused by a viral type selected from the group consisting of hemorrhagic viruses, respiratory viruses, gastrointestinal viruses, exanthematous viruses, hepatic viruses, cutaneous viruses, and viruses that cause neurologic disease.

Embodiment 18: The method of any one of the preceding embodiments, wherein step (a) comprises obtaining multiple samples from the built environment, wherein at least one sample is taken from a high-touch surface and at least one sample is taken from a surface in a high-occupancy area.

Embodiment 19: The method of any one of the preceding embodiments, wherein step (b) comprises performing an assay to detect the presence of at least one virus selected from the group consisting of ebola virus; dengue virus; novavirus; viruses that cause Lassa fever, yellow fever, Marburg hemorrhagic fever, and Crimean-Congo hemorrhagic fever; rhinovirus; coronavirus; adenovirus; influenza virus; parainfluenza virus; respiratory syncytial virus; enterovirus; norovirus; rotavirus; astrovirus, viruses that cause measles, rubella, chickenpox/shingles, roseola, smallpox, and fifth disease; chikungunya virus; hepatitis virus; herpesvirus, papilloma virus; molluscum contagionsum; polio virus; rabies virus; and viruses that cause viral meningitis and encephalitis.

Embodiment 20: The method of embodiment 19, wherein the virus is selected from the group consisting of: H1N1, H1N2, H2N2, H2N3, H3N1, H3N2, H3N8, H5N1, H5N2, H5N3, H5N6, H5N8, H5N9, H6N1, H6N2, H7N1, H7N2, H7N3, H7N4, H7N7, H7N9, H9N2, H10N7, H10N8, H11N2, H11N9, H17N10, H18N11, HPIV-1, HPIV-2, HPIV-3, HPIV-4, HAdV-B, HAdV-C, 229E, OC43, NL63, HUK1, SARS-CoV-2, MERS-CoV, SARS-CoV, Sin Nombre orthohantavirus, Black Creek Canal orthohantavirus, Puumala virus, Thaland virus; HRV-A1, HRV-A2, HRV-A7-13, HRV-A15, HRV-A16, HRV-A18-25, HRV-A28-34, HRV-A36, HRV-A38-41, HRV-A43-47, HRV-A49-51, HRV-A53-68, HRV-A71, HRV-A73-78, HRV-A80-82, HRV-A85, HRV-A88-90, HRV-A94-96, HRV-A98, HRV-A100-103, HRV-B3-6, HRV-B14, HRV-B17, HRV-B26, HRV-B27, HRV-B35, HRV-B37, HRV-B42, HRV-B48, HRV-B52, HRV-B69, HRV-B70, HRV-B72, HRV-B79, HRV-B83, HRV-B84, HRV-B86, HRV-B91-93, HRV-B97, HRV-B99, and HRV-C1-51.

Embodiment 21: The method of any one of the preceding embodiments, wherein step (c) comprises determining the amount of human-associated microbes present on said surface from the one or more microbiome features indicative of one or more human-associated microbes, wherein the one or more microbiome features comprising one or more DNA and/or RNA sequences.

Embodiment 22: The method any one of the preceding embodiments, wherein step (c) comprises determining the presence and relative abundance of at least one human-associated microbe selected from the group consisting of: Actinomyces, Aerococcus, Akkermansia, Alistipes, Alloiococcus, Anaerococcus, Anaerotruncus, Atopobium, Bacteroides, Barnesiella, Bifidobacterium, Blautia, Butyrivibrio, Chlamydia, Clostridium, Corynebacterium, Cutibacterium (formerly Propionibacterium), Dialister, Dysgonomonas, Enterobacter, Enterococcus, Escherichia, Faecalibacterium, Fusobacterium, Gardnerella, Gemella, Haemophilus, Klebsiella, Kocuria, Lactobacillus, Lactococcus, Megasphera, Methanobrevibacter, Micrococcus, Mobiluncus, Moraxella, Mycobacterium, Mycoplasma, Neisseria, Oxalobacter, Papillibacter, Parabacteriodes, Parvimonas, Peptoniphilus, Peptostreptococcus, Porphyromonas, Prevotella, Pseudomonas, Roseburia, Ruminococcus, Sneathia, Spirochaeta, Staphylococcus, Streptococcus, Villonella, Alternaria, Aspergillus, Candida, Cladosporium, Curvularia, Embellisia, Fusarium, Penicillium, Saccharomyces, Stachybotrys, Thermomyces, Trichophyton, Malassezia, and Rhodotorula.

Embodiment 23: The method of any one of the preceding embodiments, further comprising an additional step of determining the relative or total amount of human DNA on the surface from the one or more physical samples prior to performing step (d).

Embodiment 24: The method of any one of the preceding embodiments, further comprising performing an ATP test step on said one or more physical samples prior to performing step (d).

Embodiment 25: The method of any one of the preceding embodiments, wherein the level of risk assigned is high if any infectious agent of step (b) is identified.

Embodiment 26: The method of any one of the preceding embodiments, wherein the level of risk assigned is intermediate if step (b) indicates no infectious agent and step (c) indicates a level of human-associated microbes that meets or exceeds a predetermined threshold.

Embodiment 27: The method of any one of the preceding embodiments, wherein the level of risk assigned is low if step (b) indicates no infectious agent and step (c) indicates a level of human-associated microbes of below a predetermined threshold.

Embodiment 28: The method of any one of the preceding embodiments, wherein one or more of said obtaining, determining, generating and assigning steps are performed by a robot at or near the location of the built environment.

Embodiment 29: The method of embodiment 28, wherein the robot provides the risk-level assignment(s) of step (d) as a data output.

Embodiment 30: The method of embodiment 28 or 29, wherein said obtaining step is remotely guided by a human.

Embodiment 31: The method of embodiment 28, wherein the robot uses an Artificial Intelligence algorithm to iterate its sampling location pattern within the built environment based on the risk-level assignment(s) of step (d).

Embodiment 32: The method of any one of embodiments 28-31, wherein the robot's data output is operably linked to a network of the built environment such that areas that pose immediate risk upon detection are visibly and/or audibly marked until an intervention occurs.

Embodiment 33: A method of determining if one or more interventions to alter indoor environmental quality are needed to reduce infection risk to humans within a built environment, the method comprising: (a) obtaining a one or more physical samples from at least one surface in the built environment; (b) determining from the one or more physical samples if one or more infectious agents are present on the at least one surface; and (c) generating a microbiome signature for the at least one surface from the one or more physical samples and determining from the signature the amount of human-associated microbes present on said surface; and (d) if: (i) any infectious agent of step (b) is identified on said surface, determining that a high-level intervention should be performed; or (ii) step (b) indicates no infectious agent and step (c) indicates a level of human-associated microbes that meets or exceeds a predetermined threshold, determining that a low-level intervention should be performed; or (iii) step (b) indicates no infectious agent and step (c) indicates a level of human-associated microbes of below a predetermined threshold, determining that no intervention is needed.

Embodiment 34: The method of embodiment 33, wherein the intervention is selected from the group consisting of applying a disinfecting agent to the surface, applying a cleaning agent to the surface, applying a disinfecting agent and a cleaning agent to the surface, restricting access to humans until cleaning and/or disinfecting is performed, restricting access to humans until a certain amount of time has passed, modifying a surface, exposing the surface to ultraviolet light, altering cleaning protocols such as increasing frequency of cleaning and/or identity of cleaning and/or disinfecting agents, altering one or more environmental parameters, increasing the ratio of outdoor:indoor air introduced into the building through an air handling system and increasing air flow to at least 2 air changes per hour.

Embodiment 35: The method of embodiment 33, wherein an infectious agent of step (b) is identified on said surface or step (c) indicates a level of human-associated microbes that meets or exceeds a predetermined threshold, and if: (i) any infectious agent of step (b) is identified on said surface, the method additionally comprises causing a high-level intervention to be performed; or (ii) step (b) indicates no infectious agent and step (c) indicates a level of human-associated microbes of that meets or exceeds a predetermined threshold, the method additionally comprises causing a low-level intervention to be performed.

Embodiment 36: The method of embodiment 33 or embodiment 35, wherein the high-level intervention comprises restricting access to humans until cleaning and/or disinfecting is performed and/or restricting access to humans until a certain amount of time has passed.

Embodiment 37: The method of any one of embodiments 33-35, wherein the low-level intervention comprises applying a disinfecting agent to the surface, applying a cleaning agent to the surface, applying a disinfecting agent and a cleaning agent to the surface, modifying a surface, exposing the surface to ultraviolet light, altering cleaning protocols such as increasing frequency of cleaning and/or identity of cleaning and/or disinfecting agents, reagents, and altering one or more environmental parameters.

Embodiment 38: The method of any one of embodiments 33-36, further comprising performing an ATP test on said surface prior to performing step (d).

Embodiment 39: The method of any one of embodiments 33-37, wherein the built environment is selected from the group consisting of residential, institutional, commercial, health care, and transportation environments.

Embodiment 40: The method of any one of embodiments 33-39, wherein the infection is selected from the group consisting of bacterial, viral, fungal, protozoal, and parasitic infections.

Embodiment 41: The method of embodiment 40, wherein the infection is selected from the group consisting of hemorrhagic viruses, respiratory viruses, gastrointestinal viruses, exanthematous viruses, hepatic viruses, cutaneous viruses, and viruses that cause neurologic disease.

Embodiment 42: The method of any one of embodiments 33-41, wherein step (a) further comprises obtaining multiple samples from the built environment, wherein at least one sample is taken from a high-touch surface and at least one sample is taken from a surface in a high-occupancy area.

Embodiment 43: The method of any one of embodiments 33-42, wherein step (b) comprises performing an assay to detect the presence of at least one virus selected from the group consisting of ebola virus; dengue virus; novavirus; viruses that cause Lassa fever, yellow fever, Marburg hemorrhagic fever, and Crimean-Congo hemorrhagic fever; rhinovirus; coronavirus; adenovirus; influenza virus; parainfluenza virus; respiratory syncytial virus; enterovirus; norovirus; rotavirus; astrovirus, viruses that cause measles, rubella, chickenpox/shingles, roseola, smallpox, and fifth disease; chikungunya virus; herpesvirus, hepatitis virus; and molluscum contagionsum.

Embodiment 44: The method of embodiment 43, wherein the virus is selected from the group consisting of: H1N1, H1N2, H2N2, H2N3, H3N1, H3N2, H3N8, H5N1, H5N2, H5N3, H5N6, H5N8, H5N9, H6N1, H6N2, H7N1, H7N2, H7N3, H7N4, H7N7, H7N9, H9N2, H10N7, H10N8, H11N2, H11N9, H17N10, H18N11, HPIV-1, HPIV-2, HPIV-3, HPIV-4, HAdV-B, HAdV-C, 229E, OC43, NL63, HUK1, SARS-CoV-2, MERS-CoV, SARS-CoV, Sin Nombre orthohantavirus, Black Creek Canal orthohantavirus, Puumala virus, Thaland virus; HRV-A1, HRV-A2, HRV-A7-13, HRV-A15, HRV-A16, HRV-A18-25, HRV-A28-34, HRV-A36, HRV-A38-41, HRV-A43-47, HRV-A49-51, HRV-A53-68, HRV-A71, HRV-A73-78, HRV-A80-82, HRV-A85, HRV-A88-90, HRV-A94-96, HRV-A98, HRV-A100-103, HRV-B3-6, HRV-B14, HRV-B17, HRV-B26, HRV-B27, HRV-B35, HRV-B37, HRV-B42, HRV-B48, HRV-B52, HRV-B69, HRV-B70, HRV-B72, HRV-B79, HRV-B83, HRV-B84, HRV-B86, HRV-B91-93, HRV-B97, HRV-B99, and HRV-C1-51.

Embodiment 45: The method of any one of embodiments 33-44, wherein step (c) comprises determining the determining the amount of human-associated microbes present on said surface from the one or more microbiome features indicative of one or more human-associated microbes, wherein the one or more microbiome features comprising one or more DNA and/or RNA sequences.

Embodiment 46: The method of embodiment 45, wherein step (c) comprises determining the presence and relative abundance of human-associated microbes selected from the group consisting of: Actinomyces, Aerococcus, Akkermansia, Alistipes, Alloiococcus, Anaerococcus, Anaerotruncus, Atopobium, Bacteroides, Barnesiella, Bifidobacterium, Blautia, Butyrivibrio, Chlamydia, Clostridium, Corynebacterium, Cutibacterium (formerly Propionibacterium), Dialister, Dysgonomonas, Enterobacter, Enterococcus, Escherichia, Faecalibacterium, Fusobacterium, Gardnerella, Gemella, Haemophilus, Klebsiella, Kocuria, Lactobacillus, Lactococcus, Megasphera, Methanobrevibacter, Micrococcus, Mobiluncus, Moraxella, Mycobacterium, Mycoplasma, Neisseria, Oxalobacter, Papillibacter, Parabacteriodes, Parvimonas, Peptoniphilus, Peptostreptococcus, Porphyromonas, Prevotella, Pseudomonas, Roseburia, Ruminococcus, Sneathia, Spirochaeta, Staphylococcus, Streptococcus, Villonella, Alternaria, Aspergillus, Candida, Cladosporium, Curvularia, Embellisia, Fusarium, Penicillium, Saccharomyces, Stachybotrys, Thermomyces, Trichophyton, Malassezia, and Rhodotorula.

Embodiment 47: The method of any one of embodiments 33-46, wherein step (c) further comprises an additional step of determining the relative or total amount of human DNA on the surface from the one or more physical samples.

Embodiment 48: The method of any one of embodiments 33-47, further comprising performing an ATP test step on said one or more physical samples prior to performing step (d).

Embodiment 49: The method of any one of embodiments 33-48, wherein at least one of said obtaining, determining, generating and assigning steps are performed by a robot at or near the location of the built environment.

Embodiment 50: The method of embodiment 49, wherein the robot provides the intervention level(s) of step (d) as a data output.

Embodiment 51: The method of any one of embodiments 33-50, wherein said obtaining step is remotely guided by a human.

Embodiment 52: The method of embodiment 49, wherein the robot uses an Artificial Intelligence algorithm to repeat its sampling location pattern within the built environment based on the determinations of step (d).

Embodiment 53: The method of embodiments 33-52, wherein the robot's data output is operably linked to a network of the built environment such that areas that pose immediate risk upon detection are visibly and/or audibly marked until an intervention occurs.

Embodiment 54: A method of determining the effectiveness of an intervention to alter risk of infection in a built environment, the method comprising: (a) obtaining a one or more physical samples from at least one surface in the built environment; (b) determining from the one or more physical samples the amount of one or more infectious agents, if present on the at least one surface; (c) generating a microbiome signature for the at least one surface from the one or more physical samples and determining from the signature the number and quantity of species of human-associated microbes present on said surface; (d) performing at least one intervention on said surface; (e) repeating steps (a)-(c) with respect to said surface; and (f) determining if the amount of infectious agent(s) of step (b) and the number and/or quantity of human-associated microbes of step (c) is reduced.

Embodiment 55: The method of embodiment 54, wherein the intervention is selected from the group consisting of applying a disinfecting agent to the surface, applying a cleaning agent to the surface, applying a disinfecting agent and a cleaning agent to the surface, restricting access to humans until cleaning and/or disinfecting is performed, restricting access to humans until a certain amount of time has passed, modifying a surface, exposing the surface to ultraviolet light, altering cleaning protocols such as increasing frequency of cleaning and/or identity of cleaning and/or disinfecting agents, reagents, and altering one or more environmental parameters.

Embodiment 56: A method of assessing the risk of infection to humans from a human contact object, the method comprising: (a) obtaining a one or more physical samples from at least one surface of the high-contact object; (b) determining from the one or more physical samples if one or more infectious agents are present on the at least one surface; (c) generating a microbiome signature for the at least one surface from the one or more physical samples and determining from the signature the amount of human-associated microbes present on said surface; and (d) based on the results of steps (b) and (c), assigning a level of risk of infection to one or more humans for touching said high-contact object or any sub-area of said high-contact object.

### BRIEF DESCRIPTION OF THE DRAWINGS

FIG. 1: One embodiment of a decision tree used to assign sample-level scores based on the presence of SARS-Cov2, the total bacterial load (pg, from qPCR analyses), the relative proportions of human-associated taxa (HAT), and the relative proportion of a subset of important HATs.

### DETAILED DESCRIPTION

### Definitions

Terms used in the claims and specification are defined as set forth below unless otherwise specified.

“Human contact objects” refer to objects that are not physically attached to a built environment and regularly contacted by humans. Examples include personal effects and high-touch objects.

“Personal effects” refer to objects associated with a person, such as, e.g., mobile phones, key chains, wallets, wallet contents (e.g., credit cards), purses, water bottles, eye glasses, clothing, shoes, jewelry, toys, appliances, tools, leashes, dining ware, and food storage containers.

“High-touch objects” means objects that a human comes directly into contact with, particularly when multiple humans come into contact with the object on a regular basis. Examples include packages, products on shelves at stores, grocery bags, shopping carts, shopping baskets, common-use scooters and bicycles, a clipboard in a doctor's office, retail and courier touch screens.

“Intervention” means altering the identity and/or abundance of microbes occupying a surface or circulating in air by using physical, chemical, biological, and/or radiological means. Examples include wiping surfaces with an alcohol solution, applying soapy water and/or bleach, applying a probiotic cleaner, exposure with ultraviolet light, washing and drying bedding, linens, and/or clothes, allowing time to pass before allowing human occupancy or human touching, increasing the air flow rate and/or air changes per hour, increasing the amount of outdoor air entering the building, and increasing the amount of filtration by introducing additional filters and/or reducing the pore size of filters. Interventions can also include changing the a density of humans in the building per square foot and requiring use of personal protective equipment (PPE), such as masks.

“Built environment” means an environment intended to host humans that has been constructed by humans and is not a natural environment. Examples of built environments are homes, buildings, aircraft, ships, submarines, subway stations, rail cars, automobiles, space stations, bunkers, spacecraft, office buildings, commercial buildings, dormitories, prisons and hospitals. Built environments can be residential (e.g., multiunit, single-family home), institutional (e.g., dormitory, prison, elder care), commercial (e.g., office building, warehouse, distribution center, factory), health care (e.g., hospital), or transportation (e.g., aircraft, cruise ship, bus) environments.

“Sub-area means” a subset of an entire area. For example, a sub-area of an office building can be a bathroom or a particular door handle. A sub-area of a bicycle can be the handle.

“Infectious agent” means any agent that is capable of infecting a human or other mammal and causing illness. Examples of infectious agents include bacteria, archaea, viruses, prions, protozoa, algae, fungi, and parasites.

“Microbiome signature” means composite fingerprint (e.g. a genetic (genome-based) and/or metabolic signature) showing the identity/presence (e.g., genus and/or species) and optionally the relative abundance of one or more types of microbe from a particular environment or object. Microbiome signatures can be obtained through sequencing or PCR, including qPCR. For example, qPCR to detect Firmicutes, Actinobacteria, and Proteobacteria provides a microbiome signature that reveals the level of human breathing in a built environment since these are non-pathogenic microbes that colonize the human respiratory tract and are entrained in the air exiting the human body during exhalation.

“Metabolic signature” means composite fingerprint containing one or more elements of the metabalome, proteome, and/or metatrascriptome, showing the identity and relative abundance of one or more types of microbe(s) from a particular environment or object.

“Human-associated microbes” are microbes, such as bacteria, archaea, fungi and viruses, that are known to colonize the human body. Human-associated microbes are found in and on many parts of the human body. Sub-classes of microbes are known to colonize particular areas of the human body and form specific microbiomes such as the human oral, nasal, fecal, vaginal, and skin microbiomes. Humans shed approximately 30 million microbes per hour off of skin and hair and through exhaling, sneezing, and coughing microbes entrained in the air exiting the respiratory system. Aerosolized droplets that are expelled from the human body during a sneeze are filled with human-associated microbes, including, e.g., pathogens such as influenza virus particles, if the human is infected and contagious. The presence of human-associated microbes is an indicator of potential for the presence of infectious agents.

“High-touch surface” means surface in a built environment that is regularly contacted by humans. Examples include doorknobs, cabinet handles, toilet handles, elevator buttons, retail touch screens, faucets, desk surfaces, computer keyboards, factory sewing machines, factory assembly line elements, factory tools, and retail store conveyor belts.

“High-occupancy area” means an area in a built environment that is occupied at higher human density per square foot by humans and/or occupied more often than other areas on a relative scale within a built environment and/or between built environments. Examples are common areas such as bathrooms, communal kitchens, high density cubicle areas, elevators, and classrooms.

The phrase “assigning a level of risk of infection to one or more humans” refers to any determination that narrows the full spectrum of risk of infection that is possible (e.g., no risk of infection to infection is inevitable). The assignment of level of risk of infection can be noted (e.g., in a paper and/or computer record), communicated (e.g., via a computer display as the output of an analysis), or may simply be implicit in a determination that a particular level of intervention (e.g., to lower the risk of infection) is appropriate or that no intervention is needed because the risk of infection is negligible.

As used herein, the term “network” refers to a system having linked computers or having one or more computers linked to one or more control elements. Networks typically include some form of display, whereby data and/or results of analyses can be presented to a user. Networks include LANs (Local Area Networks), PANs (Personal Area Networks), MANs (Metropolitan Area Networks), and WANs (Wide Area Networks).

As used with respect to a network, a “control element” is any element that, directly or indirectly, effects a change in a condition in a built environment.

“Modifying a surface” means making a qualitative change to a surface (i.e., making a change to a surface other than a change in treatment of the surface). Examples of modifying a surface include removing carpeting, applying a coating, and replacing one type of material (e.g., plastic) with another type (e.g., steel).

As used herein with respect to microbes (e.g., infectious agents or human-associated (or other) microbes), the terms “amount” or “quantity” refer to any parameter indicative of the amount of microbes present, e.g., on a surface or in the air. In some embodiments, the determination of the amount of microbes present in a sample or on a surface or in the air can include a determination of quantity, types, and relative abundance of microbial groups, or human-associated microbial groups that are present. Types of microbial groups can be determined using taxonomic, phylogenetic, functional, or other classifications. Examples of taxonomic groups can include families, genera, species, or strains. Examples of other classifications include human-associated taxa (HAT, e.g., microbes present in or on the human body) and infectious agents. Quantity can refer to the total amount of microbial genomic DNA, microbial DNA, viral DNA, viral RNA, or the total amount of microbial organisms, microbial genes, sequence reads, genetic features, microbial cells, microbial load, or biomass present on a surface or in the air, or the amount of any of these elements within one or more microbial groups. The relative abundance, proportion, or relative proportion of a microbial group can include the ratio of one microbial group quantity relative to total amount of microbial quantity, or the proportion of one microbial group quantity to another.

### Assessing the Risk of Infection to Humans

In General

The present disclosure provides a method of assessing the risk of infection to humans. This method is applicable to a variety of contexts. For example, the method can be applied to assessing the risk of infection to humans in a built environment or an object, such as a high-contact object.

The method entails obtaining a physical sample from at least one surface or from the air in the built environment or from at least one surface of an object (e.g., high-contact object). This method can be employed with respect to a surface or air sample in any built environment, numerous examples of which are provided in PCT Publication No. 2015171834 (published on Nov. 12, 2015), which is incorporated by reference herein for this description. The physical sample is assayed for the presence of one or more infectious agents. In some embodiments, this assay is quantitative, allowing the determination of the amounts of the one or more infectious agents present. This determination indicates if one or more infectious agents are present (and, optionally, their amount[s]) on the surface.

A microbiome signature indicative of the surface or air is also generated from the physical sample. The signature includes one or more features of one or more hum-associated microbes. This allows the determination of the amount of one or more human-associated microbes present on the surface or in the air.

As explained in more detail below, the results of the assay for the presence and or amounts of one or more infectious agents, taken with the results of the determination of the amount of one or more human-associated microbes, enables assigning a level of risk of infection to one or more humans for occupying the built environment or any sub-area of the built environment (e.g., the sub-area containing the surface) or, as the case may be, assigning a level of risk of infection to one or more humans for touching the high-contact object or any sub-area of the high-contact object. The results of the assay can also assign a level of risk for a class of objects without having sampled all of such objects. For example, a plurality (e.g., 1,000) boxes containing merchandise for return to a retailer can be sampled immediately upon arrival to determine the average level of risk for humans handling such boxes immediately upon arrival. This information can inform the retailer of which operational modifications, if any, should be made to the procedure for handling future arriving boxes.

In some embodiments, the method includes performing an ATP test step on a physical sample obtained from the surface prior to assigning a level of risk of infection. The ATP test provides a way of rapidly measuring actively growing cells, including microbes, though detection of adenosine triphosphate (ATP). Since ATP is usually only present in living cells, the amount of ATP detected on a surface or in a physical sample obtained from the surface provides an indication of the number of living cells on the surface or the “bioburden” of the surface. Methods of testing for ATP are well known, and a number of ATP tests are available commercially. Some, such as the Hygiena SystemSURE PLUS™ ATP Measurement System (ATP Meter), are devices that provide an ATP measurement from a test swab. In some embodiments, the result of an ATP test is factored into the assignment of the level of risk of infection. A higher reading in the ATP test would tend to weigh in favor of a higher level of risk.

In some embodiments, the method includes obtaining multiple samples from a built environment, wherein at least one sample is taken from a high-touch surface and/or at least one sample is taken from a surface in a high-occupancy area or sub-area. In some embodiments, the method includes obtaining multiple air samples from a built environment by air samplers distributed throughout the building or by sampling air in a central air handling system that is representative of all the air in the building.

Obtaining Physical Samples

Samples can be obtained by any technique not materially altering or destroying the molecules needed for the analysis (e.g., nucleic acids) that may be contained therein. For example, a surface can be sampled by swabbing with a sterile cotton or nylon swab. Material picked up from the surface can then be rinsed from the swab with sterile solution. Sampling can be automated at recurring or programmed intervals or can be responsive to a specific event, such as a change in a facility or business operation.

In some embodiments, the physical sample can be obtained by a robot, which can be affixed in the built environment or mobile. There is a wealth of experience with using robots to collect physical samples that can be adapted to sampling surfaces in built environments or on objects. Examples include extraterrestrial sampling robots (see, e.g., Zhang et al. (2019) Nature Astronomy 3:487-497) and autonomous systems that improve environmental sampling at sea (Matheson, MIT News, Nov. 4, 2019). In some embodiments, a sampling robot that is remotely guided by a human is employed. In other embodiments, the robot is autonomous.

Air can be sampled to assess the quality, type, identity, metabolic profile, allergenicity, and gene content for various target molecules. Air samples may be obtained via various well-known techniques in the art, including but not limited to passive settling dish assays (empty, sterile petri plate), passive static-charged cloth assays, and vacuum air pump collection using at least one of a sterile button filter (such as SKC celllose membrane filters), a sterile filter cup (such as Nalgene Polypropylene Analytical Test Filter Funnel), and a liquid impinge (such as SKC BioSampler). Air can also be sampled by sampling a filter that has been or is in the flow of air through an air-handling system such as an HVAC system. For example, a filter or other permeable or semi-permeable substrate can be inserted for a certain period of time into the flow of air in an HVAC system that is returning from inside the built environment, then removed and sampled. Microbes can be released from the substrate by rinsing or immersing the substrate in a sterile buffer, then performing qPCR, PCR, or sequence analysis on the liquid.

Passive samplers can be used to collect particles and bioaerosols that settle out during the sampling period. Passive samplers are generally inexpensive and thereby greatly reduce the cost and the need for infrastructure in a facility or on the sampling location. Passive samplers are semi-disposable and, due to their low cost, can be employed in great numbers, allowing for a better cover and more data being collected. In some instances, passive samplers are small in size and may be easily hidden, and thereby lower the risk of disturbance. Non-limiting examples of passive sampling devices include sterile petri plates, diffusive gradients in thin films (e.g. DGT samplers), Chemcatcher, Polar organic chemical integrative samplers (POCIS), and air sampling pumps.

Following the sampling period, the passive samplers are collected and the target molecules are collected from the sampling devices. In some instances, the target molecule samples are collected by swabbing one or more surfaces of the passive samplers. In other instances, the target molecules are collected by washing one or more surfaces of the passive sampler with a small volume of liquid buffer solution. The collected samples are then suspended in a buffer solution until further laboratory processing.

Static-charged cloths collect target molecules, including cells and bioaerosols, by static attraction. Following the sampling period, the target molecules are extracted from the static-charged cloths by i) dissolving the static-charged cloth in a buffer solution; ii) washing the static-charged cloth in buffer solution; or iii) washing the static-charged cloth in a charged buffer solution to release the target molecules from the cloth.

Vacuum-drawn air samplers typically include a porous air filter coupled to a vacuum air pump. Air is drawn through the filter and target particles (e.g., cells or molecules) larger than the pore size of the filter settle on the filter. Filter pore size may vary depending upon the desired target. In some instances, a vacuum-drawn air sampler is selected having a filter pore size from 0.2 μm diameter to 5 μm diameter, wherein the vacuum-drawn air sampler is used to collect, e.g., bacterial cells and/or fungal cells.

Determining the Presence or Quantity of Infectious Agents

Any convenient means for determining the presence of and, optionally quantifying, one or more infectious agents in the sample can be employed in the methods described herein. Standard methods include protein-based methods, such as immunoassays, and nucleic acid-based methods.

In some embodiments, the characterization of samples typically involves identification of nucleic acids in the sample. The sample can be subjected to a process to extract nucleic acid, which can be DNA or RNA. Sequence analysis can be performed by determining the nucleotide sequence of all nucleic acid in the sample or by some portion thereof. Sequence analysis can be performed by hybridization to a probe or an array of probes. Sequence analysis can also be performed by nucleic acid sequencing. Sequencing of RNA can be used as an indicator of viability of the cells to determine which cells in the sample were living or dead at the time of sampling. Nucleic acids can also be characterized sufficient to type associated microbes by hybridization assay or selective amplification, such as by RFLP analysis, PCR analysis, STR analysis, Illumina sequencing and AmpFLP analysis, and the like.

Although any form of sequencing can be used, so called next-generation or massively parallel methods offer considerable advantages over traditional Sanger and Maxam-Gilbert sequencing. Some next generation sequence methods amplify by emulsion PCR. A target nucleic acid immobilized to beads via a target capture oligomer provides a suitable starting material for emulsion PCR. The beads are mixed with PCR reagents and emulsion oil to create individual micro-reactors containing single beads (Margulies et al., Nature 437, 376-80 (2005)). The emulsion is then broken, and the individual beads with amplified DNA are sequenced. The sequencing can be pyrosequencing performed for example using a Roche 454 GS FLX sequencer (454 Life Sciences, Branford, Conn. 06405). Alternatively, sequencing can be ligation/detection performed for example using an ABI SOLiD Sequencing System (Life Technologies, Carlsbad, Calif. 92008). In another variation, target nucleic acids are eluted from the target capture oligomer and immobilized in different locations on an array (e.g., the HiScanSQ (Illumina, San Diego, Calif. 92121)). The target nucleic acids are amplified by bridge amplification and sequenced by template-directed incorporation of labeled nucleotides, in an array format (Illumina). In another approach, target nucleic acids are eluted from the target capture oligomer, and single molecules are analyzed by detecting in real-time the incorporation nucleotides by a polymerase (single-molecule real-time sequencing or SMRT sequencing). The nucleotides can be labeled nucleotides that release a signal when incorporated (e.g., Pacific Biosciences, Eid et al., Sciences 323 pp. 133-138 (2009)) or unlabeled nucleotides, wherein the system measures a chemical change on incorporation (e.g., Ion Torrent Personal Genome Machine (Guilform, Conn. 94080)).

High-throughput screening methods generally use robotics, data processing and control software, liquid handling devices, and sensitive detectors to rapidly conduct high numbers of, e.g., genetic tests. High-throughput technologies and methods are capable of screening approximately 10 million samples per hour.

The sequence analysis can be targeted to specific DNA or RNA sequences, such as those associated with rRNA, such as 23S rRNA or 16S rRNA, which can be used to identify which species/genus/taxa of microbes are present and the relative abundance of each.

The sequence analysis can be a metagenomic approach, which sequences all DNA captured by the sampling methods. This may be done with or without an amplification step. This provides information about not only which species/genus/taxa of microbe is present and its relative abundance, but also which known pathogenic genes, antibiotic resistance, and indicator genes are present in the sample. Metagenomic sequencing provides a much richer data set than amplifying markers such as 16S rRNA and allows for greater insight into the microbiome of the built environment or object. Because microbes evolve far faster than multicellular organisms, they are constantly adapting to new environmental conditions and can undergo natural selection to allow a much higher degree or proliferation and colonization of a facility over time, such as adapting to more effectively colonize a particular surface. Genetic changes that allow such adaptation are captured by metagenomic sequencing but not by amplifying and sequencing only a specific marker region.

PCR amplification can also be used to generate microbiome signatures. For example, primers specific to certain microbes know to colonize the human body, e.g., the human respiratory tract, can be used to obtain a microbiome signature that is indicative of the amount of microbes known to colonize the human respiratory tract. Examples are Firmicutes, Actinobacteria, Proteobacteria, Staphylococcus epidermidis, viridans group streptococci (VGS), Corynebacterium spp. (diphtheroids), Propionibacterium spp., Haemophilus spp. Prevotella, Fusobacterium, Moraxella, Candida, Pseudomonas, Streptococcus, Prevotella, Fusobacterium, and Veillonella. qPCR determines relative amounts of each amplification target, and together the amplified targets constitute a human-associated microbiome signature. Higher levels of the preceding microbes can be indicative of one or more conditions that are conducive to transmission of human respiratory pathogens such as poor ventilation, high density of humans, and ventilation that is recycling air without introducing any or enough outside air to dilute or remove airborne microbes.

Infectious Agents

Any infectious agent for which a test, or a basis for designing at test (e.g., nucleic acid sequence), exists can be determined, and optionally quantified, in the methods described herein. Generally, the sample is assayed for one or more infectious agents that are known or suspected to infect humans. In some embodiments, one or more of the infectious agents are suspected to be present in the built environment or on the object from which the sample is taken. In certain embodiments, one or more of the infectious agents are suspected to be circulating in the vicinity of the built environment or in an environment to which the object has been exposed. In some embodiments, the infectious agents are known human pathogens. Examples include bacteria, archaea, viruses, prions, protozoa, algae, fungi or, and parasites.

Illustrative infectious agents of particular interest include bacteria, such as organisms from the families, genera, and/or species: Streptococcus, Corynebacterium, Flavimonas, Lactobacillus, Burkholderia, Bacillus, Bradyrhizobium, Propionibacterium, Enterobacter, Neisseria, Pseudomonas, Streptococcus, Staphylococcus, Nictrospumilus, Nitrosospira, Nitrosomonas, Kytococcus sedentarius, Staphylococcus epidermidis, Staphylococcus haemolyticus, Ralstonia pickettii, Enterobacter, Kocuria rhizophila, Micrococcus luteus, Microcystis aeruginosa, Prochlorococcus marinus, Methylocella silvestris, Methylobacterium extorquens, Pseudomonas, Staphylococcus spidermidis, Enterococcus faecalis, Klebsiella, Propionibacterium, Micrococcaceae, Bacteriodaceae, Methylobacterium, Sphingomonas, Mycobacterium, Pseudomonas aeruginosa, Legionella, Mycobacterium, Sphingomonas, Propionibacterineae, Xanthomonadaceae, Micrococcineae, Sphingomonas, Caenibacterium, and Enterobacteriaceae.

Viral infectious agents of interest with respect to the present methods include any viruses that infect humans. Of particular interest are those that cause or predispose humans to disease, such as, for example, hemorrhagic viruses, respiratory viruses, gastrointestinal viruses, exanthematous viruses, hepatic viruses, cutaneous viruses, and viruses that cause neurologic disease. Illustrative hemorrhagic viruses include ebola virus, dengue virus, novavirus, and viruses that cause Lassa fever, yellow fever, Marburg hemorrhagic fever, and Crimean-Congo hemorrhagic fever. Illustrative respiratory viruses include rhinovirus, coronavirus, adenovirus, influenza virus, parainfluenza virus, hanta virus (e.g., orthohantavirus), and respiratory syncytial virus. Illustrative gastrointestinal viruses include enterovirus, norovirus, rotavirus, adenovirus, and astrovirus. Illustrative exanthematous viruses include viruses that cause measles, rubella, chickenpox/shingles, roseola, smallpox, and fifth disease, as well as chikungunya virus. Illustrative hepatic viruses include hepatitis virus (e.g., hepatitis A-E). Illustrative cutaneous virus include herpesvirus, papilloma virus, and molluscum contagionsum. Illustrative viruses that cause neurological disease include those polio virus, rabies virus, and viruses that cause viral meningitis and encephalitis.

Specific examples of viruses of interest with respect to the present methods include the following: for influenza virus: H1N1, H1N2, H2N2, H2N3, H3N1, H3N2, H3N8, H5N1, H5N2, H5N3, H5N6, H5N8, H5N9, H6N1, H6N2, H7N1, H7N2, H7N3, H7N4, H7N7, H7N9, H9N2, H10N7, H10N8, H11N2, H11N9, H17N10, and H18N11; for parainfluenza virus: parainfluenza viruses 1-4 (denoted HPIV-1, HPIV-2, HPIV-3, and HPIV-4); for adenovirus: HAdV-B and HAdV-C; for coronavirus: 229E, OC43, NL63, HUK1, SARS-CoV-2, MERS-CoV, and SARS-CoV; for othorhantavirus: Sin Nombre orthohantavirus, Black Creek Canal orthohantavirus, Puumala virus, and Thaland virus. Human rhinovirus serotype names are of the form HRV-Xn, where X is the rhinovirus species (A, B, or C) and n is an index number. Species A and B have used the same index, while Species C has a separate index. Valid index numbers for rhinoviruses of interest with respect to the present methods are as follows: rhinovirus A: 1, 2, 7-13, 15, 16, 18-25, 28-34, 36, 38-41, 43-47, 49-51, 53-68, 71, 73-78, 80-82, 85, 88-90, 94-96, 98, and 100-103; rhinovirus B: 3-6, 14, 17, 26, 27, 35, 37, 42, 48, 52, 69, 70, 72, 79, 83, 84, 86, 91-93, 97, and 99; and rhinovirus C: 1-51.

Determining Human-Associated Microbe Amounts from Microbiome Signatures

To determine amount(s) of one or more human-associated microbes, a sample obtained from air or a surface in a built environment or a surface of an object is analyzed for the presence and amount of one or more microbiome features that indicate the presence and amount of one or more human-associated microbes. In some embodiments, the features are genetic features (e.g., particular sequences). Nucleic acids can be analyzed as described above to provide the features of the microbiome signatures for the surface or air. The microbiome signature can include features from DNA, RNA, or both.

The generation of microbiome signatures is discussed extensively in PCT Publication No. 2015171834 (published on Nov. 12, 2015), which is incorporated by reference herein for this description. In particular, this publication states that once samples are obtained, they are analyzed to provide a characterization of the microbiome. The characterization typically involves identification of nucleic acids in the sample by sequence analysis. Analysis can also be performed by PCR methods, including qPCR. Alternatively or additionally, collected target molecules may be used to count cells, i.e., to infer the number of cells present, and, as noted above, whole cells can be collected, and if collected to ensure viability maintained, even to grow viable cells in culture.

In some embodiments in which only a subset of the nucleic acids in a sample are characterized, the sequence analysis may be targeted to specific DNA or RNA sequences, such as those associated with 23S rRNA or 16S rRNA, which can be used to identify which species/genus/taxa of microbes are present and the relative abundance of each; those associated with antibiotic resistance genes (see Liu and Pop. ARDB-Antibiotic Resistance Genes Database. Nucleic Acids Res. 2009 January; 37 (Database issue): D443-7); or those associated with indicator genes, which are genes associated with improved performance of the system or reduced performance of the system and which may or may not have a known function. In some embodiments, the antibiotic resistance genes or other indicator genes themselves are sequenced, either as part of a metagenomic sequencing or as amplified products. In some embodiments, the sequence identification step will involve the determination of whether any nucleic acid sequences associated with an indicator taxa is present. An “indicator taxa” refers to a microbe whose presence or absence is of particular relevance to the analysis being conducted.

In some embodiments, metagenomics is performed, such that all of the DNA (or all of the nucleic acid or all of the RNA) from a sample is sequenced. This may be done with or without an amplification step, but in many instances, there will be no amplification step. This provides information about not only which species/genus/taxa of microbe is present and its relative abundance, but also which genes, known or unknown, are present. Sequences identified in a microbiome sample are checked for identity comparison to predetermined sequences. Levels of identity of 80% or higher are generally considered by those skilled in the art to be indicative of a sequence encoding the same or similar biochemical function. Algorithms for analyzing raw sequence data from samples can set the desired level of identity, such as greater than 50%, greater than 60%, greater than 70%, greater than 80%, or greater than 90% identity to any one of a set of predetermined sequences in a reference database.

For the purposes of the methods described herein, the microbiome signature can be one that is simply representative of the air or a surface (e.g., it may be generated without any prior knowledge of the genera or species of microbes present on the surface, and it may thus include features that are not particularly relevant to the methods described herein). A representative microbiome signature can be generated, for example, by using primers designed to amplify all nucleic acids in the sample (e.g., random primers). In some embodiments, microbiome signature generation includes analyzing the nucleic acid from the sample for one or more specific features found in one or more human-associated microbes, for example, one or more predetermined nucleic acid sequences, which can be, e.g., from 23S rRNA and/or 16S rRNA from one or more human-associated bacteria or from ITS genomic regions found in human-associated fungi. In certain embodiments, microbiome signature generation can include analyzing the nucleic acid from the sample only for one or more specific features found in one or more human-associated microbes (in addition to suitable at least one control sequence that indicates that nucleic acid analysis worked properly).

Human-associated microbes include, e.g., bacteria, archaea, fungi, and viruses, that are known to colonize the human body. Human-associated microbes are found in and on many parts of the human body. Sub-classes of microbes are known to colonize particular areas of the human body and form specific microbiomes such as the human oral, nasal, fecal, vaginal, and skin microbiomes. In various embodiments, generating a microbiome from the surface or air sample can include analyzing the nucleic acids for features from microbes making up one or any combination of such microbiomes (e.g., the human oral, nasal, fecal, vaginal, and skin microbiomes.) In some embodiments, for a comprehensive analysis, generating a microbiome from the surface sample can include analyzing the nucleic acids for microbes representative of all of the human oral, nasal, respiratory, fecal, vaginal, and skin microbiomes. The analysis can be tailored to the context in which it is being carried out, such as, for example, analyzing the nucleic acids for features of the human fecal microbiome, such as crAssphage, when the sample is from a surface in a bathroom. The features analyzed can be from pathogenic human-associated microbes, though this is not a requirement of this step of the method. Generally, it is advantageous to generate a microbiome including features of human-associated microbes that serve as an indicator of the potential for the presence of infectious agents, even, in some embodiments, before they are present.

After generating a microbiome signature for the air or a surface, as explained above, one can determine from the signature the amount of human-associated microbes present in the air or on the surface. How this determination is carried out can vary depending on what is included in the microbiome signature. If the microbiome signature is one that is simply representative of the air or surface, the determination includes identifying features of human-associated microbes and quantifying them. For example, one can use qPCR to determine the amount of features indicative of human-microbes, relative to the amount of all features in the microbial signature. If the microbiome signature is specific to human-associated microbes, it can be sufficient to determine the amount the absolute amount of one or more features obtained from the sample (which can then be normalized to a control).

Human-Associated Microbes

Illustrative human-associated microbes include, but are not limited to human-associated bacteria and human-associated fungi. Examples of human-associated bacteria of interest with respect to the present methods include: Actinobacteria, Actinomyces, Aerococcus, Akkermansia, Alistipes, Alloiococcus, Anaerococcus, Anaerotruncus, Atopobium, Bacteroides, Barnesiella, Bifidobacterium, Blautia, Butyrivibrio, Candida, Chlamydia, Clostridium, Corynebacterium, Cutibacterium (formerly Propionibacterium), Dialister, Dysgonomonas, Enterobacter, Enterococcus, Escherichia, Faecalibacterium, Firmicutes, Fusobacterium, Gardnerella, Gemella, Haemophilus, Klebsiella, Kocuria, Lactobacillus, Lactococcus, Megasphera, Methanobrevibacter, Micrococcus, Mobiluncus, Moraxella, Mycobacterium, Mycoplasma, Neisseria, Oxalobacter, Papillibacter, Parabacteriodes, Parvimonas, Peptoniphilus, Peptostreptococcus, Porphyromonas, Prevotella, Propionibacterium, Proteobacteria, Pseudomonas, Roseburia, Ruminococcus, Sneathia, Spirochaeta, Staphylococcus, Streptococcus, and Villonella. Examples of human-associated fungi of interest with respect to the present methods include: Alternaria, Aspergillus, Candida, Cladosporium, Curvularia, Embellisia, Fusarium, Penicillium, Saccharomyces, Stachybotrys, Staphylococcus epidermidis, Thermomyces, Trichophyton, Malassezia, and Rhodotorula, Veillonella, and viridans group streptococci (VGS).

Assigning a Level of Risk of Infection

Detection or lack of detection of one or more infectious agents and various types of analysis of the human-associated taxa found in each sample enables the assignment of the level of risk faced by humans occupying or transiting through the built environment or coming into contact with the object, as the case may be. In some embodiments, the level of risk is binary, i.e.: risk or lack of risk is determined. Practitioners of the methods described herein can assign risk according to numerous parameters, including those described in Example 1. In the illustrative embodiments below, the risk level can be divided into a high level of risk, an intermediate level of risk, or a low level of risk.

If any infectious agent is identified in the determination described above, the highest level of risk is assigned. This is the case regardless of the determination as to the amount of human-associated microbes that are determined.

In some embodiments, if no infectious agent is identified in the determination described above, and the amount of human-associated microbes meets or exceeds a predetermined threshold, an intermediate (not the highest and not the lowest) level of risk is assigned. In other words, the methods described here do not indicate a present risk of infection from a known infectious agent, since no infectious agent was identified, but the results indicate a high-touch surface or object or an environment with air containing a higher amount of human-associated microbes that is susceptible to transmission of infection.

In some embodiments, additional factors can be taken into account in assigning a level of risk, such as the transmission potential of a surface or air, which can relate to the number of people who touch a surface and/or the frequency with which the surface is touched or the number of people breathing in the room. In some embodiments a high transmission potential can elevate a low level of risk to a high level of risk (see, e.g., Example, Room and Building Level Scoring, Room Risk). In some embodiments, when assessing the risk associated with a particular room or with a building as a whole, multiple samples are generally taken from multiple locations and/or multiple rooms and the results from these multiple samples/rooms can be taken into account in assigning room- or building-level risk (see, e.g., id.). In some embodiments building level risk is determined by sampling the air in the return flow of an air handling system.

If no infectious agent is identified in the determination described above, and the amount of human microbes is below the predetermined threshold, a low level of risk is assigned.

The predetermined threshold of human-associated microbes is set at a level such that, above that level, the risk of infection is high enough to warrant some intervention. The level may vary depending upon the context in which the method is carried out. For example, with regard to a surface contacted by the public, the threshold may be set lower than in a context where a surface is contacted only by a few people, since the risk of spreading infection is higher in the public context. Also, if the context includes risk of infection by an easily transmissible virus that has significant health consequences (such as the risk of severe acute respiratory syndrome from SARS-CoV-2 and related viruses), the threshold may be set lower than it otherwise might be. The appropriate threshold for a given context may be determined, e.g., by testing for amount of human-associated microbes on one or more surfaces in an environment or one or more objects immediately after cleaning and/or for various times thereafter and/or after various levels of human contact. The threshold may then be set a level appropriate for that context, which can take into account factors such as the feasibility and cost of cleaning or restricting contact with people, and the benefits of such interventions, relative to the risk of infection.

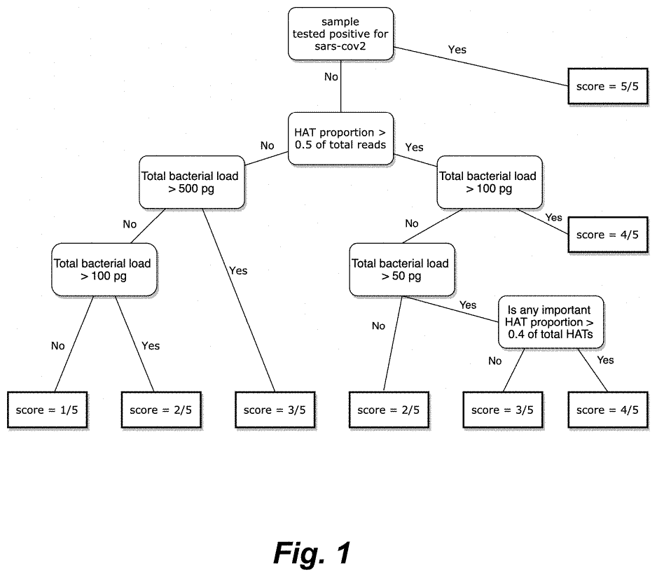

In one embodiment, illustrated in Example 1, each physical sample receives a risk score ranging from 1 to 5, where 5 means the sample was collected from a surface or from air with the highest risk of infectious disease transmission and 1 means the sample was collected from a surface or air with low risk of infectious disease transmission. The scoring takes into account four variables: a) presence of SARS-Cov2, b) total bacterial load (pg, from qPCR analyses), c) relative proportions of HATs, and d) relative proportions of a subset of important HATs. For this example, the subset of important HATs includes: Bifidobacterium, Enterococcus, Staphylococcus, Blautia, Corynebacterium, Cutibacterium, Propionibacterium, Haemophilus, and Faecalibacterium. In this embodiment, the risk score is assigned following a decision tree shown in FIG. 1 and described in Example 1.

Computer Implementation

Many of the steps involved the disclosed methods can be implemented on a suitably-programmed computer. Such steps include particularly storing and comparing microbiomes, as well as storing databases of relevant information. The computer can also be programmed to provide recommended interventions (discussed below) and to predict the effect of such changes infection risk levels. The computer can also be programmed to operate monitoring equipment used to monitor a microbiome and to receive data from such equipment characterized the microbiome. The computer can also be programmed to operate facility equipment such as HVAC systems, for example by modifying the ratio of interior to exterior air that is circulated throughout the structure.

A computer system can include a bus which interconnects major subsystems such as a central processor, a system memory, an input/output controller, an external device such as a printer via a parallel port, a display screen via a display adapter, a serial port, a keyboard, a fixed disk drive, and an internet connection. Many other devices can be connected such as a scanner, via I/O controller, and a mouse connected to serial port or a network interface. Other devices or subsystems may be connected in a similar manner. Also, it is not necessary for all of the described devices to be present to practice the methods present described herein, as discussed below. The devices and subsystems may be interconnected in different ways. Source code to implement the present disclosure may be operably disposed in system memory or stored on storage media such as a fixed disk, compact disk or the like. The computer system can be a mainframe, PC, table or cell phone, among other possibilities.

Robotics

Sampling robotics have been discussed above. In addition to sampling, one or more robots can perform one or more (or all) of the steps of determining the presence or amount of infectious agent(s), generating a microbiome and determining the amount of human-associated microbe(s), and assigning of infection risk level. The robot is typically at or near the location of the built environment or object to be tested and can be autonomous or remotely guided by a human.

In some embodiments the robot provides a risk-level assignment as a data output. In particular embodiments, the robot's data output is operably linked to a network of the built environment such, that areas that pose immediate risk upon detection are visibly and/or audibly marked until an intervention occurs.

An algorithm, such as an artificial intelligence (AI) algorithm can be employed to use risk-level assignments to determine and, e.g., repeat a sampling location pattern (optionally including frequency of sampling) within the built environment. This algorithm can be run in any convenient location (e.g. on a computer that is part of a network or on the internet), including in the robot itself.

### Determining Interventions

In some embodiments, the method entails determining suitable interventions based on the results of the method described above, i.e., based on the risk assessment level identified. In some embodiments, if the risk of infection is identified as high, a determination is made that a high-level intervention should be performed. If an intermediate risk of infection is identified, a determination is made that a low-level intervention should be performed. If a low risk of infection is identified, a determination is made that no invention need be performed. Generally, the intervention level ranges from low to high based on the degree to which the intervention is necessary to reduce the likelihood of human infection. The classification of interventions as high or low can depend upon the particular context of the intervention, in a way that balances disruption and cost on the one hand, and benefit from reducing risk of infection on the other.

Types of Interventions

In the case of built environments, inventions can include the changing of a facility operation that can be changed is the cleaning system of a facility. Such a system encompasses a number of subsidiary operation parameters, such as the chemicals used, the surfaces cleaned, the method of cleaning, and the frequency of cleaning. Sterilization procedures (for hospitals in particular, which often use UV light and/or chemicals to clean) also represent key operation parameters. Frequency and duration of sterilization using a device such as portable room disinfection systems that use pulsed xenon ultraviolet light to destroy viruses, bacteria, mold, fungus and bacterial spores in the patient environment that cause healthcare associated infections is a changeable facility operation parameter (see U.S. Ser. Nos. 13/706,926 and 13/156,131). Devices and surfaces can be introduced to reduce transmission or dissemination of microbes, such as contamination control mats.

Another type of facility operation change is a modification of one or more surfaces. Examples include changes to the type of surfaces (e.g. carpet versus hard floor and composition, e.g., fiber, wood, linoleum) present in a facility. All surfaces (ceiling, floors, walls, doors, and doorknobs and handles, equipment surfaces, ceiling tiles, paint, furniture composition, fabric, carpet, computers, light switches, personal electronic devices, bed linens, baseboards, wainscoting, materials inside wall cavities, hand drying devices and the like) in a facility, their location(s) and their relative abundance can be changed.

Further changes can be made in lighting, both in placement of lights and types (incandescent, LED, fluorescent, natural lighting).

Further changes that can be made include changes to the plumbing. The plumbing includes the nature and location of pipes and faucets and the like that deliver and remove fluids from the structure.

Other illustrative facility operation changes are changes in the operation of business including the number of workers per square foot in the structure, the hours of operation of the business, the zones within the structure used for particular functions, such as bathrooms, kitchens, and storage of products. Other changes include the turnover and flow of human occupants. Other changes include changes to the contractors, suppliers or customers of a business operating in the facility.

In some embodiments, a facility operation change can include changing the heating, ventilation, and/or air conditioning (HVAC) system. Such a system has a number of subsidiary operation parameters, such as airflow rate, filtration, and facility filter pore size (as well as the frequency of changing filters), temperature and temperature fluctuations of the air, and the relative humidity of the air. Mechanical ventilation and natural ventilation can both be used in a facility. Displacement ventilation can also be used. One measure of the operation of an HVAC system is the number of air changes per hour (total volume of the facility that is changed over by the ventilation system per unit time).

In some embodiments the intervention(s) can involve making changes to the way the air handling system is operated. The HVAC system of a facility will often allow the manager of the facility to alter the temperature, humidity, air supply source, air flow, and/or filtration of the air in the facility. Occupied spaces often ventilate at a rate of less than 1 air change per hour (ACH), and research shows that this is insufficient for diluting human-associated microbes in indoor air. Higher ventilation rates (e.g., at least 2, 3, 4, 5, 6, 7, 8, 9 or 10 ACH) effectively remove airborne microbes emitted from human occupants. Filtration in HVAC systems removes particulate matter from supply air sources. Office buildings often employ MERV-8 filtration, which removes most fungal spores, but not bacteria. Hospital operating rooms typically use more stringent filtration (MERV-15), which removes most bacteria from supply air. In some scenarios, such as operating rooms, more stringent filtering can improve performance, while in other buildings, such as offices surrounded by green space, unfiltered outdoor air would improve performance. Many HVAC systems are operated in a way that recycles air such that no air from the outdoors is introduced into the system. This mode of operation saves energy because the returning air does not need to be heated or cooled much or at all since it is already at the temperature of the building interior. Energy use can become a secondary consideration when the level of human-associated microbes is high, and/or the presence of pathogens is detected. The level of risk of infection can be reduced by increasing the ratio of outdoor:indoor air from no outside air to at least 1:5, 1:4, 1:3, 1:2, 1:1, 2:1, 3:1 4:1, 5:1 or 100% outdoor air. The combination of increasing ACH to 2 or higher and increasing the ratio of outdoor:indoor air to 1:5 or higher reduces the level of risk of infection to humans by diluting and removing human-associated microbes, including pathogens, from the built environment. A further intervention that also reduces the level of risk of infection is to reduce the density of humans allowed in the building at any given time. A further intervention that also reduces the level of risk of infection is to require humans occupying the building to wear PPE such as face masks or respirators.

As well as or instead of changing mechanical ventilation systems, the structure of the facility can be changed to introduce operable windows. These can be opened by workers as external temperature and other conditions permit. The windows can also be coupled to a monitoring system as described for the HVAC to permit their opening only when exterior conditions are overall likely to confer benefits when transferred to the interior. Other changes include the square footage of windows, the number of panes and the type of transparent material.

In particular embodiments, the intervention can include one or more of the following: applying a disinfecting agent to the surface, applying a cleaning agent to the surface, applying a disinfecting agent and a cleaning agent to the surface, restricting access to humans until cleaning and/or disinfecting is performed, restricting access to humans until a certain amount of time has passed, modifying a surface, exposing the surface to ultraviolet light, altering cleaning protocols such as increasing frequency of cleaning and/or identity of cleaning and/or disinfecting agents, reagents, and altering one or more environmental parameters such as humidity, temperature and level of ventilation. In illustrative embodiments, restricting access to humans (other than suitably protected persons whose presence is for the purpose of cleaning and/or disinfecting) until cleaning and/or disinfecting is performed, and/or restricting access to humans (other than suitably protected persons whose presence is for the purpose of cleaning and/or disinfecting) until a certain amount of time has passed are high-level interventions, whereas the following are low-level interventions: applying a disinfecting agent to the surface, applying a cleaning agent to the surface, applying a disinfecting agent and a cleaning agent to the surface, exposing the surface to ultraviolet light, altering cleaning protocols such as increasing frequency of cleaning and/or identity of cleaning and/or disinfecting agents, and altering one or more environmental parameters such as humidity, temperature and level of ventilation

Similar interventions apply to objects, except that facility operation changes relate to the conditions under which the objects are stored.

### Determining the Effectiveness of Interventions

In certain embodiments, a determination of the effectiveness of one or more interventions can be carried out. After an intervention, the analysis described above can be carried out to determine the presence, absence, and optionally the quantity, of infectious agents, and the amount of microbes, including human-associated microbes, present in a sample or on a surface in a built environment or on an object. This analysis can follow an earlier iteration of the same (or a similar) analysis (e.g., one that prompted the intervention). An intervention is deemed to be effective if a decrease in the total amount of microbial quantity, or the quantity, relative abundance, proportion, or relative proportion of a microbial group, transmission potential, the amount or relative abundance of one or more microbial group (e.g. the proportion or relative proportion of HAT) is detected relative to the earlier analysis.

If the intervention is to be deemed effective, the intervention can be performed at regular intervals to maintain the risk of infection at an acceptable level. If the intervention is not deemed to be sufficiently effective, one or more other interventions can be carried out, in addition to, or instead of, the initial intervention and their effectiveness assessed in the same manner. In this manner the benefits of each intervention, alone or in combination with others, can be assessed, which can be used to determine a suitable intervention or intervention regimen to maintain the risk of infection at an acceptably low level.

### EXAMPLES

### Example 1

There are many different environments where the risk-of-transmission (or risk-of-infection, as described above) test can be deployed. This example illustrates an office building as a use case. The combination of areas with high and low foot traffic, and multiple surfaces and materials, provided an excellent opportunity for the detection of SARS-COV-2 in the office environment, as well as for the identification and quantification of human-associated taxa (HAT), that can be used to assess the risk of transmission.

To evaluate the entire office building, we sampled multiple surfaces within each room. Samples were assayed in parallel for 2 SARS-COV-2 sequences spiked in to represent environmental viral RNA and a pan-bacterial assay, each through qPCR. Human-associated bacteria present were identified through 1×250 bp Illumina sequencing performed on the HiSeq platform. A multilevel scoring technique was used to produce the overall building risk score. In general, testing individual surfaces alone can only establish the microbial load of that surface without any indication of its impact on the room or the building. For example, doorknobs can be reservoirs of multiple microorganisms, including SARS-CoV-2, and represent surfaces that are touched by a large number of people moving in and out of a room. In addition, due to this high amount of interaction of doorknobs with human skin, they can constitute a reservoir of human-associated taxa, including possible microbial pathogens. Additional factors including Touch Rate, Touch Turnover, Surface Persistence Rate, Annual Occupied Hours, Occupant Diversity, and Ventilation are used to assess the risk of entering the building as a whole.

Sampling

Environmental samples were collected using sterile Copan nylon flocked swabs (Brescia, Italy) under dry conditions. Swabs tips were immediately immersed in MAWI DNA Microbiome (Hayward, Calif., USA) tubes after sampling. Swabs were rotated as indicated by the manufacturer and removed from the tubes. A fixed volume of these swab wash isolates were spiked with SARS-CoV-2 RNA control from Twist Biosciences (San Francisco, Calif., USA).

Extraction

Bacterial genomic DNA and viral RNA were extracted from each sample using the Lucigen MasterPure Total Nucleic Acid kit (Middletown, Wis., USA), following the manufacturer's instructions. Cell lysis was initiated by combining 500 μL of swab wash isolate with 500 μL of T&C lysis buffer (Lucigen Kit). Extracted nucleic acid was dried under nitrogen purge and samples were resuspended in 100 μL of TE buffer.

SARS-CoV-2 RT qPCR Detection

Primer and probe sets were obtained from IDT, (Coralville, Iowa, USA). Assay sequences used are provided in Table 1. Single target amplification reactions were carried out using 54 of extracted sample with Luna Reverse Transcription qPCR (RT-qPCR) kits from New England Biolabs (Ipswitch, Mass., USA), per manufacturer's instructions. The thermocycling conditions for the reaction consisted of a) reverse transcription for 10 minutes @ 55° C., b) activation for 1 minute @ 95° C. and c) 40 cycles of denaturation for 10 seconds (95° C.) and extension for 30 seconds (60° C.) and were performed on an Applied Biosystems QuantStudio 6 (South San Francisco, Calif., USA) at 20 μL volumes in a 384-well plate.

TABLE 1
2019 SARS-CoV-2 Primer and Probe Designs

| Target Name | Forward Primer Sequence | Reverse Primer Sequence | Probe Sequence |
|---|---|---|---|
| 2019- | 5′- | 5′- | 5′-FAM- |
| nCoV_N1 | GACCCCAAAATCAGCGAAA | TCTGGTTACTGCCAGTTGAATCT | ACCCCGCATTACGTTTGGTGGACC- |
| | T-3′ | G-3′ | BHQ1-3′ |
| | (SEQ ID NO: 1) | (SEQ ID NO: 3) | (SEQ ID NO: 5) |
| 2019- | 5′-TTACAAACATTGGCCGCAA | 5′-GCGCGACATTCCGAAGAA- | 5′-FAM-ACAATTTGCCCCCAGCGCTTCAG- |
| nCoV_N2 | A-3′ | 3′ | BHQ1-3′ |
| | (SEQ ID NO:2) | (SEQ ID NO: 4) | (SEQ ID NO: 6) |

Pan-Bacterial qPCR Detection

Sequences

Pan Bacterial 16s:

a. Forward:
(SEQ ID NO: 7)
5′ -TCCTACGGGAGGCAGCAGT-3′
b. Reverse:
(SEQ ID NO: 8)
5′ -GGACTACCAGGGTATCTAATCCTGTT-3′
c. Probe:
(SEQ ID NO: 9)
(FAM)-5′- CGTATTACCGCGGCTGCTGGCAC3′-(TAMRA)

This assay was designed with a long amplicon length (467 bases) to increase coverage of species based on the 16S assay from Nadkarni, Mangala A., et al., “Determination of bacterial load by real-time PCR using a broad-range (universal) probe and primers set” (2002) Microbiology 148.1:257-266.

Thermocycling conditions were:

Activation for 2 minutes @ 95° C.; and 40 cycles of denaturation for 15 seconds (95° C.) and extension for 30 seconds (60° C.), where the plate was read by the thermocycler.

16sV4 Amplification and Library Preparation

16sV4 libraries were prepared using techniques adapted from Caparoso, et. al., Proc. Natl. Acad. Sci. USA (Mar. 15, 2011) 108(1):4516-4522. Briefly; samples were PCR amplified for 30 cycles with the 515f and 806r primers shown below. Following gel electrophoresis QC, 2 μL of this PCR product was used as the input for a further 6 cycles of PCR using primers which incorporated the Illumina sequencing adaptors as well as the Golay barcodes with the reverse primers. These PCR products were cleaned of primer dimers using standard mag-bead clean-up, and their concentration was determined using quant-it reagents (Thermo-Fisher) in 96-well plate format on a Molecular Devices (CA) fluorescence plate reader. Libraries exhibiting concentrations over three times the median concentration were normalized.

PCR1
515f:
(SEQ ID NO: 10)
5′ GTGCCAGCMGCCGCGGTA 3′
806r:
(SEQ ID NO: 11)
5′ GACTACHVGGGTWTCTAAT 3′
PCR2
Forward:
(SEQ ID NO: 12)
AATGATACGGCGACCACCGAGATCTACACTATGGTAATTGTGTGCCAGCMG
CCGCGGTAA

Reverse Primer construct (68 bases from 5 components) (1 example below)
1) Reverse complement of 3′ Illumina adapter 5′ CAAGCAGAAGACGGCATACGAGAT/(SEQ ID NO:13)
2) GolayBarcode(Unique for each sample)/TCCCTTGTCTCC (SEQ ID NO:14)
3) Reverse primer Pad/AGTCAGTCAG (SEQ ID NO:15)
4) Reverse Primer Linker/CC
5) Reverse Primer/GGACTACHVGGGTWTCTAAT 3′ (SEQ ID NO:16)

Pooling and QC

Equal volumes of each library within one 96-well plate were pooled. These pools were concentrated using standard mag-bead clean-up and were analyzed using an Agilent Bioanalyzer. The pools were diluted to 10 nM and pooled into a second pool containing libraries from up to 12 96-well plates. The concentration of this pool was measured and diluted to 2 nM.

Sequencing

The 2 nM pool was denatured with sodium hydroxide and diluted to 8 pM. This pool was combined with 8 pM PhiX for a total PhiX makeup of 20%. One library pool was loaded onto each lane of a HiSeq flowcell using Illumina's cBot cluster generation system with a Rapid SR Cluster Kit. Target density was 700 k/sq.mm and runs were started using standard Illumina HiSeq2500 protocols with the exception of mixing in the following custom sequencing primers at 500 nM concentration.

Sequencing primers:

a. Forward:
(SEQ ID NO: 17)
5′ -TATGGTAATTGTGTGCCAGCMGCCGCGGTAA-3′
b. Index:
(SEQ ID NO: 18)
5′ -ATTAGAWACCCBDGTAGTCCGGCTGACTGACT-3′

Bioinformatics Pipeline

Demultiplexing

Sequence runs on NGS instruments are typically carried out with multiple samples pooled together as stated above. An index tag (also called a barcode) consisting of a unique sequence of between 6 and 12 bp was added to each sample so that the sequence reads from different samples could be identified. To associate each read to a unique sample, a demultiplexing process was carried out after sequencing, using BCL2FASTQ V2.20 (Illumina, San Diego, Calif.).

PhiX Removal

Because amplicon sequencing generates reads that start with the same nucleotide sequences, a different DNA sequence needs to be present to be able to generate signal diversity, allowing the sequencer to discriminate between clusters and create signal thresholds for base calling. PhiX was used to generate this diversity. After sequencing, PhiX sequences were removed from the sequencing data during the demultiplex process, as PhiX does not have an associated barcode attached, therefore it was not assigned to any sample.

Artifact Removal

High-throughput sequencing can generate sequencing artifacts, such as long stretches of a single nucleotides or sequences that are not real and correspond to adapters used in the sequencing reactions. Before proceeding to any in-depth analysis, these sequences need to be removed from the dataset. Cutadapt was used to achieve this. Sequences that had more than 10 repetitions of any nucleotide were removed from the dataset. In addition, each read was compared to the nucleotide sequences of the most commonly used sequencing adapters, and if the adapter sequence was present, that section was trimmed from the read. Finally, if after trimming the sequence was shorter than the established threshold (in this example, 241 nucleotides), the read was removed from the dataset.

Feature Inference

For feature inference, the bioinformatics pipeline filtered, collapsed, and aggregated all the identical reads into single unique sequences while retaining their abundances. First, the data was filtered by removing low-quality sequences and then truncated to a specific length (for example, 240 nucleotides). Then, sequences were dereplicated, collapsing exact duplicates, but keeping track of their real abundance in the dataset. Following this, reads were denoised to identify the correct biological sequences and remove errors introduced by high-throughput. All of these steps were performed using Dada2. A comprehensive report was generated to evaluate the quality of each individual sample before it was uploaded to the database.

Data Analysis

Taxonomy Identification

To obtain the taxonomic information for each feature, the nucleotide sequences were compared against a taxonomic reference database. In this example Dada2 achieved taxonomic classification using a naive Bayesian classifier method. This approach assigned the taxonomy of each feature to the best possible level (Kingdom, Phylum, Class, Order, Family, Genus). For every sample, the total read count for each taxonomic group was calculated as the sum of the read counts for the features assigned to that group. Next, the total read counts for all human-associated taxa (HAT) was quantified in each sample. Because the counts are not directly comparable between samples, due to differences in sequencing depth, the total read count for each HAT was normalized by the total number reads obtained in each sample (relative proportion). With this value, the relative proportion of each HAT and the relative proportion of non-HAT bacteria within each sample were estimated.

qPCR Limits and Calibration

Using qPCR analysis, the presence/absence of SARS-CoV-2 was established, as well as the total amount of bacterial DNA, reported in picograms of DNA (total bacterial load), present in the sample.

For a sample to be considered positive for SARS-CoC-2, both target genes (nCoV_N1 and nCoV_N2) have to be positive (<40.00 Ct) as well as the extraction control (MS2). If both target genes are negative and the extraction control is positive, the sample is considered negative for SARS-CoV-2.

To quantify the amount of bacterial DNA present in a sample, a qPCR assay was performed using pan-bacterial primers targeting the 16S rRNA region (described in the laboratory methods). Also, a standard curve was obtained by performing dilutions (7 dilutions, between 0.128 to 2000 picograms) of a standard target DNA. This curve was used to transform the Ct values obtained for the bacterial DNA to a mass in picograms. To quantify the total amount of DNA for each individual HAT, the proportion of reads assigned to each taxon was multiplied by the total bacterial load in the sample, obtaining a mass in picograms for each bacterial taxon. If the value of the mass is below 0.1 picograms (the limit of detection of the assay), the corresponding mass was considered to be 0 picograms.

Sample Level Scoring

In one embodiment, each sample receives a risk score ranging from 1 to 5, where 5 means the sample was collected from a surface with the highest risk of infectious disease transmission and 1 means the sample was collected from a surface with low risk of infectious disease transmission. The scoring takes into account four variables: a) presence of SARS-Cov2, b) total bacterial load (pg, from qPCR analyses), c) relative proportions of HATs, and d) relative proportions of a subset of important HATs. For this example, the subset of important HATs includes: Bifidobacterium, Enterococcus, Staphylococcus, Blautia, Corynebacterium, Cutibacterium, Propionibacterium, Haemophilus, and Faecalibacterium.

In this embodiment, the risk score is assigned following a decision tree (FIG. 1). The cutoff values at each decision node are calculated from a reference database of ˜5,000 indoor surface samples of bacterial communities collected throughout the world. In this example, the total bacterial load, the proportion of HATs, and the proportion of specific HATs on different surfaces is analyzed to define a set of rules. Other embodiments can utilize a different set of rules in determining a risk score. Within this dataset, 75% of the samples have a relative proportion of 0.5 HAT or less within a sample. In this example, this 75-percentile of the sample set establishes 0.5 HAT as the threshold when a sample is considered high in HATs. Therefore, if the total proportion of HATs within a sample is higher than 0.5, it is considered a sample high in HATs. Regarding the total bacterial load, the variation in bacterial load (picograms of DNA) in samples from this particular dataset averaging across different kinds of surfaces are analyzed. The 75th (˜500 pg DNA), 50th (˜100 pg DNA), and 25th percentile (50 pg) of the bacterial load are used as thresholds for high, medium, and low bacterial load in the decision tree. The variation in the relative proportions of each important HAT mentioned above are analyzed. In this particular database, only 10% of the samples have a relative proportion of 0.4 or greater of these important HATs within a sample, and thus 0.4 is used as the cutoff for that node.

Room and Building Level Scoring

While the score from a single sample estimates the risk of infection to humans for touching the surface from which the sample was taken, it does not provide a risk assessment of the entire built environment that was sampled to a human for occupying or transiting through the environment. There are additional factors that may influence the total risk of transmission to an individual or occupant human. Below is one embodiment for the process of scaling individual sample scores to room-level risk, and then room risk scores to building-level risk.

Room Risk

For any individual surface, the “Transmission Potential” to a new person is a combination of both how often it is touched and how many, or what fraction of the occupants, touch it in a day. One embodiment of calculating Room Risk is as follows:

Touch Rate (TR)=frequency of touches per day (normalized)

Touch Turnover (TT)=fraction of the occupant population touching it per day

Surface Persistence Rate (SPR)=length of time pathogens persist on surface type

Transmission Potential(TP)=TR*TT * SPR(TP ranges from 0 to 1)

The realized transmission risk for a particular surface (the “Surface Risk”), is a function of both its Transmission Potential and the total bacterial load of that surface (i.e., the Sample Score (from the previous section). Note that the Sample Score includes all sources of human microbiome transmission to a surface (e.g. touching, breathing, coughing, spitting, sneezing etc.):

Surface Risk(SR)=Transmission Potential(TP)*Sample Score(SS)

The Room Risk of an individual room, where multiple surfaces were sampled, is determined as follows. Room Risk is the maximum Surface Risk of all samples taken from that room. One positive Sars-Cov2 sample (SS=5) results in High Room Risk. In the absence of a positive Sars-Cov-2 sample, if one sample in the room had both a high HAT load and that surface has high Transmission Potential (e.g., 100% of the people in the room touch it), then the room would also be deemed High Risk.

| | | Transmission | |
|---|---|---|---|
| Surface Type | Sample Score | Potential | Surface Risk |
| TV Button | 5 | 0.01 | 0.05 |
| Remote | 1 | 0.8 | 0.8 |
| Door Knob | 1 | 1.0 | 1 |
| Door Knob 2 | 1 | 1.0 | 1 |
| Table Surface | 4 | .9 | 3.6 |
| Whiteboard markers | 4 | .5 | 2 |

Building Risk

To calculate the risk of the building, one must take into account the Environmental Risk to provide weighting for each room in the building. For example, if only one of ten rooms is deemed high risk, but 90% of the employees spend time in that room every day, and the room is poorly ventilated, then the entire building is deemed high risk. One embodiment of calculating Building Risk is as follows:

Annual Occupied Hours (AOH)=low (0.1), medium (0.5), high (1.0)

Occupant Diversity (OD)=low (0.1), medium (0.5), high (1.0)

Ventilation (V)=No Ventilation(1.5), Mechanical (1.3), Window (1) or Both (1.1)

Environmental Factor(EF)=V*AOH*OD(EF ranges from 0.01 to 1.5)

Room Score(RS)=Room Risk*EF

Annual Occupied Hours and Occupant Diversity is calculated using the approach outlined in Kembel et al. 2014 (Kembel, Steven W., et al. (2014) “Architectural Design Drives the Biogeography of Indoor Bacterial Communities” PLoS ONE 9(1):e87093).

The Building Risk where multiple rooms were sampled, is determined as follows. One Room with Room Risk of 5 results in High Building Score. Otherwise, the Building Risk is the maximum Room Score of the remaining rooms.

| | | Environmental | |
|---|---|---|---|
| Room | Room Risk | Factor | Room Score |
| Boardroom | 5 | 1 | 5 |
| Bathroom 1 | 4 | 1.3 | 5.2 |
| Bathroom 2 | 4 | 1 | 4 |
| Entrance | 3 | 0.01 | 0.03 |

In this scenario, the Boardroom was flagged to have Room Risk of 5, which implies that one of the high touch surfaces has SARS-CoV-2 and as a result, the entire building is deemed high risk. If the Boardroom was not SARS-CoV-2 positive, then the Building would still be High Risk because Bathroom 1 was found to have a high human microbial load on surfaces with high Transmission Potential (Room Risk 4); and that room has high Annual Occupied Hours, high Occupant Diversity, and poor ventilation (Room Score 5.2). The recommended intervention for the office is to evacuate the building and to use UV light to disinfect the boardroom since where COVID-19 was found on one of the surfaces.

### REFERENCES

1. PCT Publication No. 2015171834 (published on Nov. 12, 2015)
2. Nadkarni, Mangala A., et al. (2002) “Determination of bacterial load by real-time PCR using a broad-range (universal) probe and primers set,” Microbiology 148.1:257-266.
3. Kembel, Steven W., et al. (2014) “Architectural Design Drives the Biogeography of Indoor Bacterial Communities,” PLoS ONE 9(1):e87093.
4. Caparoso, et. al., Proc. Natl. Acad. Sci. USA (Mar. 15, 2011) 108(1):4516-4522.

1. A method of assessing the risk of infection to humans in a built environment, the method comprising:
(a) obtaining one or more physical samples from a surface and/or air in the built environment;
(b) determining from the one or more physical samples if one or more infectious agents are present in the built environment;
(c) generating a microbiome signature from the one or more physical samples and determining from the signature the amount of human-associated microbes; and
(d) based on the results of steps (b) and (c), assigning a level of risk of infection to one or more humans for occupying or transiting through said built environment or any sub-area of said built environment.

2. The method of claim 1, wherein the one or more physical samples are air samples.

3. The method of claim 1, wherein the infection is selected from the group consisting of bacterial, viral, fungal, protozoal, and parasitic infections.

4. The method of claim 3, wherein the infection comprises an infection caused by a viral type selected from the group consisting of hemorrhagic viruses, respiratory viruses, gastrointestinal viruses, exanthematous viruses, hepatic viruses, cutaneous viruses, and viruses that cause neurologic disease.

5. The method of claim 4, wherein step (b) comprises performing an assay to detect the presence of at least one virus selected from the group consisting of ebola virus; dengue virus; novavirus; viruses that cause Lassa fever, yellow fever, Marburg hemorrhagic fever, and Crimean-Congo hemorrhagic fever; rhinovirus; coronavirus; adenovirus; influenza virus; parainfluenza virus; respiratory syncytial virus; enterovirus; norovirus; rotavirus; astrovirus, viruses that cause measles, rubella, chickenpox/shingles, roseola, smallpox, and fifth disease; chikungunya virus; hepatitis virus; herpesvirus, papilloma virus; molluscum contagionsum; polio virus; rabies virus; and viruses that cause viral meningitis and encephalitis.

6. The method of claim 5, wherein the virus is selected from the group consisting of: H1N1, H1N2, H2N2, H2N3, H3N1, H3N2, H3N8, H5N1, H5N2, H5N3, H5N6, H5N8, H5N9, H6N1, H6N2, H7N1, H7N2, H7N3, H7N4, H7N7, H7N9, H9N2, H10N7, H10N8, H11N2, H11N9, H17N10, H18N11, HPIV-1, HPIV-2, HPIV-3, HPIV-4, HAdV-B, HAdV-C, 229E, OC43, NL63, HUK1, SARS-CoV-2, MERS-CoV, SARS-CoV, Sin Nombre orthohantavirus, Black Creek Canal orthohantavirus, Puumala virus, Thaland virus; HRV-A1, HRV-A2, HRV-A7-13, HRV-A15, HRV-A16, HRV-A18-25, HRV-A28-34, HRV-A36, HRV-A38-41, HRV-A43-47, HRV-A49-51, HRV-A53-68, HRV-A71, HRV-A73-78, HRV-A80-82, HRV-A85, HRV-A88-90, HRV-A94-96, HRV-A98, HRV-A100-103, HRV-B3-6, HRV-B14, HRV-B17, HRV-B26, HRV-B27, HRV-B35, HRV-B37, HRV-B42, HRV-B48, HRV-B52, HRV-B69, HRV-B70, HRV-B72, HRV-B79, HRV-B83, HRV-B84, HRV-B86, HRV-B91-93, HRV-B97, HRV-B99, and HRV-C1-51.

7. The method of claim 1, wherein step (c) comprises determining the presence and relative abundance of at least one human-associated microbe selected from the group consisting of: Actinomyces, Aerococcus, Akkermansia, Alistipes, Alloiococcus, Anaerococcus, Anaerotruncus, Atopobium, Bacteroides, Barnesiella, Bifidobacterium, Blautia, Butyrivibrio, Chlamydia, Clostridium, Corynebacterium, crAssphage, Cutibacterium (formerly Propionibacterium), Dialister, Dysgonomonas, Enterobacter, Enterococcus, Escherichia, Faecalibacterium, Fusobacterium, Gardnerella, Gemella, Haemophilus, Klebsiella, Kocuria, Lactobacillus, Lactococcus, Megasphera, Methanobrevibacter, Micrococcus, Mobiluncus, Moraxella, Mycobacterium, Mycoplasma, Neisseria, Oxalobacter, Papillibacter, Parabacteriodes, Parvimonas, Peptoniphilus, Peptostreptococcus, Porphyromonas, Prevotella, Pseudomonas, Roseburia, Ruminococcus, Sneathia, Spirochaeta, Staphylococcus, Streptococcus, Villonella, Alternaria, Aspergillus, Candida, Cladosporium, Curvularia, Embellisia, Fusarium, Penicillium, Saccharomyces, Stachybotrys, Thermomyces, Trichophyton, Malassezia, and Rhodotorula.

8. The method of claim 2, wherein step (c) comprises performing qPCR to determine the presence of one or more human respiratory tract microbes selected from the group consisting of Firmicutes, Actinobacteria, Proteobacteria, Staphylococcus epidermidis, viridans group streptococci (VGS), Corynebacterium spp. (diphtheroids), Propionibacterium spp., Haemophilus spp. Prevotella, Fusobacterium, Moraxella, Candida, Pseudomonas, Streptococcus, Prevotella, Fusobacterium, and Veillonella.

9. The method of claim 1, wherein the level of risk assigned is high if any infectious agent of step (b) is identified, and one or more interventions are selected from the group consisting of introducing unfiltered outdoor air into the built environment, venting air to outdoors instead of recycling, increasing the ratio of indoor:outdoor air, increasing the air flow, and reducing occupant density.

10. The method of claim 9, wherein the air flow is increased to at least 2, 3, 4, 5, 6, 7, 8, 9 or 10 air changes per hour.

11. The method of claim 1, wherein the level of risk assigned is intermediate if step (b) indicates no infectious agent and step (c) indicates a level of human-associated microbes that meets or exceeds a predetermined threshold.

12. The method of claim 1, wherein the level of risk assigned is low if step (b) indicates no infectious agent and step (c) indicates a level of human-associated microbes below a predetermined threshold.

13. The method of claim 1, wherein the level of risk of infection determines which if any interventions are performed to reduce the level of risk.

14. A method of assessing the risk of infection to humans in a built environment, the method comprising:
(a) obtaining one or more physical samples from at least one surface in the built environment;
(b) determining from the one or more physical samples if one or more infectious agents are present on the at least one surface;
(c) generating a microbiome signature for the at least one surface from the one or more physical samples and determining from the signature the amount of human-associated microbes present on said surface; and
(d) based on the results of steps (b) and (c), assigning a level of risk of infection to one or more humans for occupying or transiting through said built environment or any sub-area of said built environment.

15-17. (canceled)

18. The method of claim 1, wherein step (a) comprises obtaining multiple samples from the built environment, wherein at least one sample is taken from a high-touch surface and at least one sample is taken from a surface in a high-occupancy area.

19. The method of claim 1, wherein step (b) comprises performing an assay to detect the presence of at least one virus selected from the group consisting of ebola virus; dengue virus; novavirus; viruses that cause Lassa fever, yellow fever, Marburg hemorrhagic fever, and Crimean-Congo hemorrhagic fever; rhinovirus; coronavirus; adenovirus; influenza virus; parainfluenza virus; respiratory syncytial virus; enterovirus; norovirus; rotavirus; astrovirus, viruses that cause measles, rubella, chickenpox/shingles, roseola, smallpox, and fifth disease; chikungunya virus; hepatitis virus; herpesvirus, papilloma virus; molluscum contagionsum; polio virus; rabies virus; and viruses that cause viral meningitis and encephalitis.

20. The method of claim 19, wherein the virus is selected from the group consisting of: H1N1, H1N2, H2N2, H2N3, H3N1, H3N2, H3N8, H5N1, H5N2, H5N3, H5N6, H5N8, H5N9, H6N1, H6N2, H7N1, H7N2, H7N3, H7N4, H7N7, H7N9, H9N2, H10N7, H10N8, H11N2, H11N9, H17N10, H18N11, HPIV-1, HPIV-2, HPIV-3, HPIV-4, HAdV-B, HAdV-C, 229E, OC43, NL63, HUK1, SARS-CoV-2, MERS-CoV, SARS-CoV, Sin Nombre orthohantavirus, Black Creek Canal orthohantavirus, Puumala virus, Thaland virus; HRV-A1, HRV-A2, HRV-A7-13, HRV-A15, HRV-A16, HRV-A18-25, HRV-A28-34, HRV-A36, HRV-A38-41, HRV-A43-47, HRV-A49-51, HRV-A53-68, HRV-A71, HRV-A73-78, HRV-A80-82, HRV-A85, HRV-A88-90, HRV-A94-96, HRV-A98, HRV-A100-103, HRV-B3-6, HRV-B14, HRV-B17, HRV-B26, HRV-B27, HRV-B35, HRV-B37, HRV-B42, HRV-B48, HRV-B52, HRV-B69, HRV-B70, HRV-B72, HRV-B79, HRV-B83, HRV-B84, HRV-B86, HRV-B91-93, HRV-B97, HRV-B99, and HRV-C1-51.

21. The method of claim 1, wherein step (c) comprises determining the amount of human-associated microbes present on said surface from the one or more microbiome features indicative of one or more human-associated microbes, wherein the one or more microbiome features comprising one or more DNA and/or RNA sequences.

22. (canceled)

23. The method of claim 1, further comprising an additional step of determining the relative or total amount of human DNA on the surface from the one or more physical samples prior to performing step (d).

24. The method of claim 1, further comprising performing an ATP test step on said one or more physical samples prior to performing step (d).

25. The method of claim 1, wherein the level of risk assigned is high if any infectious agent of step (b) is identified.

26. The method of claim 1, wherein the level of risk assigned is intermediate if step (b) indicates no infectious agent and step (c) indicates a level of human-associated microbes that meets or exceeds a predetermined threshold.

27. The method of claim 1, wherein the level of risk assigned is low if step (b) indicates no infectious agent and step (c) indicates a level of human-associated microbes of below a predetermined threshold.

28. The method of claim 1, wherein one or more of said obtaining, determining, generating and assigning steps are performed by a robot at or near the location of the built environment.

29-30. (canceled)

31. The method of claim 28, wherein the robot uses an Artificial Intelligence algorithm to iterate its sampling location pattern within the built environment based on the risk-level assignment(s) of step (d).

32. The method of claim 28, wherein the robot's data output is operably linked to a network of the built environment such that areas that pose immediate risk upon detection are visibly and/or audibly marked until an intervention occurs.

33. A method of determining if one or more interventions to alter indoor environmental quality are needed to reduce infection risk to humans within a built environment, the method comprising:
(a) obtaining a one or more physical samples from at least one surface in the built environment;
(b) determining from the one or more physical samples if one or more infectious agents are present on the at least one surface; and
(c) generating a microbiome signature for the at least one surface from the one or more physical samples and determining from the signature the amount of human-associated microbes present on said surface; and
(d) if:
(i) any infectious agent of step (b) is identified on said surface, determining that a high-level intervention should be performed; or
(ii) step (b) indicates no infectious agent and step (c) indicates a level of human-associated microbes that meets or exceeds a predetermined threshold, determining that a low-level intervention should be performed; or
(iii) step (b) indicates no infectious agent and step (c) indicates a level of human-associated microbes of below a predetermined threshold, determining that no intervention is needed.

34-53. (canceled)

54. A method of determining the effectiveness of an intervention to alter risk of infection in a built environment, the method comprising:
(a) obtaining a one or more physical samples from at least one surface in the built environment;
(b) determining from the one or more physical samples the amount of one or more infectious agents, if present on the at least one surface;
(c) generating a microbiome signature for the at least one surface from the one or more physical samples and determining from the signature the number and quantity of species of human-associated microbes present on said surface;
(d) performing at least one intervention on said surface;
(e) repeating steps (a)-(c) with respect to said surface; and
(f) determining if the amount of infectious agent(s) of step (b) and the number and/or quantity of human-associated microbes of step (c) is reduced.

55. (canceled)

56. A method of assessing the risk of infection to humans from a human contact object, the method comprising:
(a) obtaining a one or more physical samples from at least one surface of the high-contact object;
(b) determining from the one or more physical samples if one or more infectious agents are present on the at least one surface;
(c) generating a microbiome signature for the at least one surface from the one or more physical samples and determining from the signature the amount of human-associated microbes present on said surface; and
(d) based on the results of steps (b) and (c), assigning a level of risk of infection to one or more humans for touching said high-contact object or any sub-area of said high-contact object.


SEQUENCE LISTING (18 sequences)

SEQ ID NO: 1 - DNA, 20 bp, Artificial
  Primer
gaccccaaaatcagcgaaat

SEQ ID NO: 2 - DNA, 20 bp, Artificial
  Primer
ttacaaacattggccgcaaa

SEQ ID NO: 3 - DNA, 24 bp, Artificial
  Primer
tctggttactgccagttgaatctg

SEQ ID NO: 4 - DNA, 18 bp, Artificial
  Primer
gcgcgacattccgaagaa

SEQ ID NO: 5 - DNA, 24 bp, Artificial
  Probe
accccgcattacgtttggtggacc

SEQ ID NO: 6 - DNA, 23 bp, Artificial
  Probe
acaatttgcccccagcgcttcag

SEQ ID NO: 7 - DNA, 19 bp, Artificial
  Primer
tcctacgggaggcagcagt

SEQ ID NO: 8 - DNA, 26 bp, Artificial
  Primer
ggactaccagggtatctaatcctgtt

SEQ ID NO: 9 - DNA, 23 bp, Artificial
  Probe
cgtattaccgcggctgctggcac

SEQ ID NO: 10 - DNA, 18 bp, Artificial
  Primer
gtgccagcmgccgcggta

SEQ ID NO: 11 - DNA, 19 bp, Artificial
  Primer
gactachvgggtwtctaat

SEQ ID NO: 12 - DNA, 60 bp, Artificial
  Primer
aatgatacggcgaccaccgagatctacactatggtaattgtgtgccagcmgccgcggtaa

SEQ ID NO: 13 - DNA, 24 bp, Artificial
  Primer
caagcagaagacggcatacgagat

SEQ ID NO: 14 - DNA, 12 bp, Artificial
  Primer
tcccttgtctcc

SEQ ID NO: 15 - DNA, 10 bp, Artificial
  Primer
agtcagtcag

SEQ ID NO: 16 - DNA, 20 bp, Artificial
  Primer
ggactachvgggtwtctaat

SEQ ID NO: 17 - DNA, 31 bp, Artificial
  Primer
tatggtaattgtgtgccagcmgccgcggtaa

SEQ ID NO: 18 - DNA, 32 bp, Artificial
  Primer
attagawacccbdgtagtccggctgactgact